(19) United States
(12) Patent Application Publication
(10) Pub. No.: US 2007/0299322 A1
(43) Pub. Date: Dec. 27, 2007

(54) Input Device, Input Method, And Electronic Device

(75) Inventors: Yasushi Miyajima, Kanagawa (JP); Yoichiro Sako, Tokyo (JP); Toshiro Terauchi, Tokyo (JP); Makoto Inoue, Kanagawa (JP); Katsuya Shirai, Kanagawa (JP); Masamichi Asukai, Kanagawa (JP); Motoyuki Takai, Tokyo (JP); Kenichi Makino, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: SONY CORPORATION, TOKYO (JP)

(21) Appl. No.: 10/579,329

(22) PCT Filed: Nov. 11, 2004
(86) PCT No.: PCT/JP04/16769

§ 371 Date: Jul. 13, 2007

(30) Foreign Application Priority Data
Nov. 18, 2004 (JP) 2003-388241
Dec. 10, 2003 (JP) 2003-412273

(51) Int. Cl. A61B 5/117

(52) U.S. Cl. 600/301

(57) ABSTRACT

The present invention is directed to a mobile telephone capable of successively acquiring bioindices. When plane surface where display screen serving to display guide display for operation and information is provided is caused to be casing front face as a position where bioindices can be successively measured without performing explicit acquiring operation if user uses the mobile telephone by ordinary way of use at the time of operation such as calling or mail input, etc., GSR sensors are provided at the side surface portion of the outer peripheral surface side, the corner portion and/or operation surface of operation input buttons of the telephone. In a non-foldable mobile telephone (1), GSR sensors (50a) are provided at side surface portions (14) of the outer peripheral surface side of the casing, and GSR sensors (50b) are provided at corner portions (15) of the outer peripheral surface side of the casing. Further, GSR sensors (50c) are provided on the operation surfaces of operation input buttons (12). In addition, the mobile telephone further comprises data processing sections for processing detection values which have been detected by respective biosensors, whereby a detection value judgment section (105) serves to select an optimum value from detection values which have been processed at the data processing sections.

### TECHNICAL FIELD

The present invention relates to an input device, an input method and an electronic equipment which are adapted for detecting bio-information (bioinformation) of user, and relates to an input device, an input method and an electronic equipment which are adapted to have ability to detect bio-information in the ordinary process of equipment use by user. Further, the present invention relates to an input device, an input method and an electronic equipment which are adapted for eliminating unevenness of detection values based on way of use of user to have ability to perform stable detection.

This Application claims priority of Japanese Patent Application No. 2003-388241, filed on Nov. 18, 2003 and Japanese Patent Application No. 2003-412273, filed on Dec. 10, 2003, the entireties of which are incorporated by reference herein.

### BACKGROUND ART

In the so-called Internet society in which communication network is used to perform transmission of information, technologies effective for more strengthening particularly information security and/or network security are required. Under such situations where information security and individual authentication which are strong or secure are required, the technology which has high value in construction of more strong or reliable security system which is above use of password and/or various encryption technologies in recent years is Biometrics Authentication. The Biometrics primarily means biometrogy, and is directed to scientific field for measuring bio-features of living thing, and there is proposed a technology in which bio-features of the human being are caused to be marker for individual discrimination to numerically express those features to collate them with registered data to thereby perform authentication of the person himself. As well known Biometrics Authentication, there is mentioned a method in which Fingerprint is caused to be authentication marker. For example, in the patent literature 1, there is proposed a technology of detecting fingerprint of user by authentication device provided at mouse. In addition to the above, bioindices such as Ear Scanner, Iris Scanning, Retinal Scanner, Speaker Verification, Palm Print or Venous Pattern, etc. may be used to perform authentication.

In general, sensor for detection of bioindex (hereinafter simply referred to as bioindex sensor) for Biometrics authentication is not required to detect biomarker at all times, but may be adapted so that detection operation can be executed at the time of staring of equipment or at the time of release of security lock. For this reason, even if sensor for fingerprint authentication is prepared (provided) at a position different from the position with which finger of user comes into contact in ordinary state, there is no problem. In actual mobile personal computer or mobile telephone in which fingerprint authentication unit is mounted, such fingerprint authentication sensor is provided at a position which is not obstacle to ordinary use rather than the position with which the finger comes into contact.

However, it is conceivable that the Biometrics technology is not only applied to authentication use purpose, but also is applied/developed with respect to various use purposes in future. As bioindex, there are mentioned, in addition to the above-described example, GSR (Galvanic Skin Reflex), Galvanic Skin Response, pulse wave, and/or body temperature, etc. In this case, not only there exist bioindices explicitly and temporarily detected as in the case of authentication use purpose, but there may be also indices required to be regularly or successively acquired at the time of ordinary use. For example, in the case of utilizing information such as Galvanic Skin Reflect, Galvanic Skin Response, pulse wave and/or body temperature, etc. of user, there was the problem that grasping portion (manner of holding) of user is changed, strength of grasp differs (varies), or a portion is sweated in the middle of measurement so that reliability and stability of values to be measured become bad.

### DISCLOSURE OF THE INVENTION

### Problems to be Solved by the Invention

An object of the present invention is to provide a novel input device, a novel input method and a novel electronic equipment which are adapted for eliminating problems that prior arts as described above have to thereby have ability to detect bio-information of user.

Another object of the present invention is to provide an input device, an input method and an electronic equipment in which if user executes use by ordinary way of use, bioindices are successively acquired even if explicit acquiring operation is not performed, and detection can be stably performed in the state where physiological index to be detected does not change depending upon difference of manner of holding or grasping force, etc. so that reliability of detection value is permitted to be enhanced.

The input device according to the present invention proposed in order to attain the above-described objects comprises: bioindex detecting means provided at a region including a holding position of the surface of a body to be operated, that user grasps in use by finger, and for successively detecting bioindices of the user through skin of the user for a time period during which the user grasps the body to be operated; and bioindex analyzing means for analyzing bioindices which have been detected by the bioindex detecting means, thus to successively detect bioindices, at the bioindex detecting means, for a time period during which the user uses the body to be operated, and to analyze, at the bioindex analyzing means, bioindices which have been detected.

As bioindex, there may be used at least one of sweating, heartbeat, pulse wave, skin temperature, Galvanic Skin Reflex, Galvanic Skin Response, MV (Micro Vibration), myoelectric potential and SPO2 (blood oxygen saturation level), or combination of these bioindices. Particularly, as bioindex detecting means, there are used detecting means for detecting Galvanic Skin Reflex or Galvanic Skin Response between predetermined two points of palm of one hand of user, pulse wave detecting means for detecting pulse wave of user, or temperature detecting means for detecting body temperature of user, and combination of these detecting means. As the temperature detecting means, there is used temperature detecting means composed of finger tip temperature detecting means provided at a contact position with which finger of user comes into contact when an equipment is grasped by finger and for detecting finger tip temperature, and palm temperature detecting means for detecting palm temperature provided at a position with which palm of user comes into contact and for detecting palm temperature.

Moreover, the electronic equipment according to the present invention includes an input unit comprising: bioindex detecting means provided within a region including a contact position of the surface of a body to be operated, with which finger comes into contact when user uses the electronic equipment, and for successively detecting bioindices of the user through skin of the user for a time period during which the user grasps the body to be operated; and bioindex analyzing means for analyzing bioindices which have been detected by the bioindex detecting means, thus to successively detect bioindices, by the bioindex detecting means of the input unit, for a time period during which the user uses the body to be operated, and to analyze, by the bioindex analyzing means, bioindices which have been detected.

Also in the electronic equipment, as bioindex, there may be used at least one of sweating, heartbeat, pulse wave, skin temperature, Galvanic Skin Reflex, Galvanic Skin Response, MV (Micro Vibration), myoelectric potential and SPO2 (blood oxygen saturation level), and combination of these bioindices.

Particularly, in an electronic equipment such that display means for displaying guide display for operation and information is provided at the surface of a casing, detecting means for detecting Galvanic Skin Reflex or Galvanic Skin Response between predetermined two points of palm of one hand of user, or temperature detecting means for detecting body temperature of user is provided at the side surface of the casing. Moreover, detecting means as described above is provided at the position where finger of user comes into contact with the surface of operation means of the electronic equipment. Further, detecting means as described above may be provided at the corner portion of the outer peripheral surface side of the casing. Here, as the temperature detecting means, there is used temperature detecting means composed of finger tip temperature detecting means provided at the position with which finger comes into contact when user grasps equipment by finger and for detecting finger tip temperature, and palm temperature detecting means provided at the position with which palm of the user comes into contact and for detecting palm temperature.

Further, in the case where pulse wave detecting means for detecting pulse wave of user is used as bioindex detecting means, the pulse wave detecting means is provided at the rear face portion with respect to the outer casing front portion in which display means for displaying guide display for operation and information is provided. Further, the outer casing rear face portion includes a detecting portion comprising a finger holding cover having internal surface shape curved so as to take substantially the same shape as finger tip shape of user, and a finger tip insertion portion formed between the finger holding cover and the outer casing rear face, and is caused to be of the structure comprising light emitting means at the internal surface of the finger holding cover, and comprising light receiving means as pulse wave detecting means at an outer casing rear face position opposite to the light emitting means.

The structure in which the detecting portion composed of finger holding cover and finger tip insertion portion formed between the finger holding cover and the rear face of the casing is provided can be also applied in the case where temperature detecting means is provided at the rear face of the casing. In this instance, the outer casing rear face of the detecting portion comprises finger tip temperature detecting means.

Another input device according to the present invention includes plural bioindex detecting means, and further comprises selector means for selecting at least one bioindex from bioindices which have been detected by the plural bioindex detecting means, and bioindex analyzing means for analyzing the bioindex which has been selected by the selector means, wherein at least one bioindex is selected, at the selector means, from bioindices which have been detected by the plural bioindex detecting means to analyze the selected bioindex.

The selector means is operative to compare signal-to-noise ratios of output values which have been detected by the plural bioindex detecting means to select an output value having a value of higher signal-to-noise ratio. Alternatively, the selector means is operative to compare detection levels of output values which have been detected by the plural bioindex detecting means to select an output value having higher detection level. Furthermore, the selector means is operative to compare auto-correlation functions of output values which have been detected by the plural bioindex detecting means to select an output value in which correlation is taken to more degree.

Moreover, at the selector means, values which have been detected substantially as the same value at plural bioindex detecting means may be selected as an output value. Further, an average value obtained by averaging values detected at respective plural bioindex detecting means may be selected as an output value.

The respective plural bioindex detecting means may be similar bioindex detecting means for detecting the same bioindex, or may be different kinds of bioindex detecting means for detecting the same bioindex by different techniques.

Moreover, as bioindex to be acquired, there is at least one of sweating, heartbeat, pulse wave, skin temperature, Galvanic Skin Reflex, Galvanic Skin Response, MV (Micro Vibration), myoelectric potential and SPO2 (blood oxygen saturation level), or combination of these bioindices.

The input device according to the present invention may be provided at personal computer, television image receiver, video and/or audio equipment, operation input unit of electronic equipment including air conditioner, and/or controller for television game machine. In addition, the input device may be provided an operation unit such as handle or control (steering) lever, etc., used when user who performs running operation in transport equipment such as automotive vehicle, train, airplane and/or ship, etc., and/or industrial equipment including industrial machine performs control (steering) operation.

The input method according to the present invention includes: plural bioindex detection steps of successively detecting, by detecting means provided within a region including a contact position of finger of the surface of a body to be operated, with which finger comes into contact when user grasps an equipment to use the equipment, bioindices of the user through skin of the user for a time period during which the user grasps, by finger, the body to be operated; a selection step of selecting at least one bioindex information from bioindex information which have been detected at the plural bioindex detection steps; and a bioindex analysis step of analyzing bioindex information which has been selected at the selection step, thus to select at least one bioindex from the bioindices which have been detected at the plural bioindex detection steps to analyze the selected bioindex.

Here, respective plural bioindex detection steps may be step of detecting the same bioindex, or may be step of detecting the same bioindex by different techniques. Here, bioindex to be detected may be at least one of sweating, heartbeat, pulse wave, skin temperature, Galvanic Skin Reflex, Galvanic Skin Response, MV (Micro Vibration), myoelectric potential and SPO2 (blood oxygen saturation level), or combination of these bioindices.

Furthermore, the electronic equipment according to the present invention includes: an input unit comprising plural bioindex detecting means provided within a region including a contact position of the surface of a body to be operated, with which finger for grasping an equipment comes into contact when user grasps the equipment in use, and for successively detecting bioindices of the user through skin of the user for a time period during which the user grasps the equipment; selector means for selecting at least one bioindex from bioindices which have been detected by the plural bioindex detecting means; and bioindex analyzing means for analyzing the bioindex which has been selected by the selector means.

In accordance with the input device, the input method and the electronic equipment according to the present invention, it is possible to successively acquire bioindices through skin surface of finger in contact with equipment when user grasps, by finger, the body to be operated for the purpose of performing operation without allowing user to explicitly execute acquiring operation of bioindex. Moreover, when user executes use by way of ordinary use, even if explicit acquiring operation is not performed, bioindices can be successively acquired, and detection can be stably performed in the state where physiological index detected is not changed depending upon difference of manner of holding or grasping force, etc. so that reliability of detection value can be enhanced. If various bioindices can be stably acquired with high reliability, new entertainment use purpose and/or new technical use purpose in which new biometrics technology of bioindex is applied can be created.

Still further objects of the present invention and practical merits which can be obtained by the present invention will become more apparent from the explanation of embodiments which will be given below with reference to the attached drawings.

### BRIEF DESCRIPTION OF THE DRAWINGS

FIG. 1 is a front view showing the state where user grasps non-foldable type mobile telephone by hand.

FIG. 2 is a front view showing the state where user grasps foldable type mobile telephone by finger.

FIG. 3 is a perspective view showing an example where sensors for detection of GSR (hereinafter simply referred to as GSR sensors) are attached to the side surface portions of the outer peripheral surface side of the non-foldable type mobile telephone.

FIG. 4 is a perspective view showing an example where GSR sensors are attached to corner portions of the outer peripheral surface side of the non-foldable type mobile telephone.

FIG. 5 is a perspective view showing an example where GSR sensors are attached to operation input buttons of the non-foldable type mobile telephone.

FIG. 6 is a view for explaining an example where GSR sensors are attached to the side surface portions of the outer peripheral surface side of the foldable type mobile telephone.

FIG. 7 is a perspective view showing an example where GSR sensors are attached to the corner portions of the outer peripheral surface side of the foldable type mobile telephone.

FIG. 8 is a perspective view showing an example where GSR sensors are attached on operation input buttons of the foldable type mobile telephone.

FIG. 9 is a perspective view showing a mobile telephone in which sensor portion for detection of pulse wave (hereinafter simply refereed to as pulse wave sensor portion) is provided.

FIG. 10A is a cross sectional view showing the pulse wave sensor portion attached to the mobile telephone in the state cut in length side direction of the mobile telephone, and FIG. 10B is a cross sectional view showing pulse wave sensor portion in the state cut in short side direction of the mobile telephone.

FIG. 11 is a perspective view showing the state where user grasps mobile telephone having pulse wave sensor.

FIG. 12 is a side view showing pulse wave sensor portion.

FIG. 13 is a block diagram showing a bioindex detecting apparatus used in the present invention.

FIG. 14 is a flowchart for explaining the processing when the bioindex detecting apparatus judges reliability of detection values at respective biosensors to select an optimum value.

### BEST MODE FOR CARRYING OUT THE INVENTION

Practical examples of the present invention will now be explained with reference to the attached drawings.

### (1) First Embodiment

Initially, the first embodiment of the present invention will be explained. In this embodiment, sensor is provided at a position where if user executes use by ordinary way of use, bioindices can be successively acquired even when explicit acquiring operation is not performed. As bioindex to be detected in this embodiment, there may be bioindex such that an electronic equipment which is a body to be operated is grasped by finger so that bioindex can be detected through skin surface. In this example, as index used in use purpose different from authentication use purpose in which operation for detection is explicitly and temporarily performed, and index required to be successively acquired at the time of ordinary use by user, there are mentioned sweating, heartbeat, pulse wave, skin temperature, Galvanic Skin Reflex, Galvanic Skin Response, MV (Micro Vibration), myoelectric potential, and SPO2 (blood oxygen saturation level), etc.

This embodiment is applied to portable or mobile type telephone (hereinafter referred to as mobile telephone) as an electric equipment, and explanation will be given by taking an example of detecting bioindices from user who utilizes the mobile telephone. The bioindices detected here are bioindices which can be relatively easily acquired, and explanation will be given by taking an example of detecting, e.g., Galvanic Skin Reflex, Galvanic Skin Response, pulse wave or body temperature (skin temperature) of user.

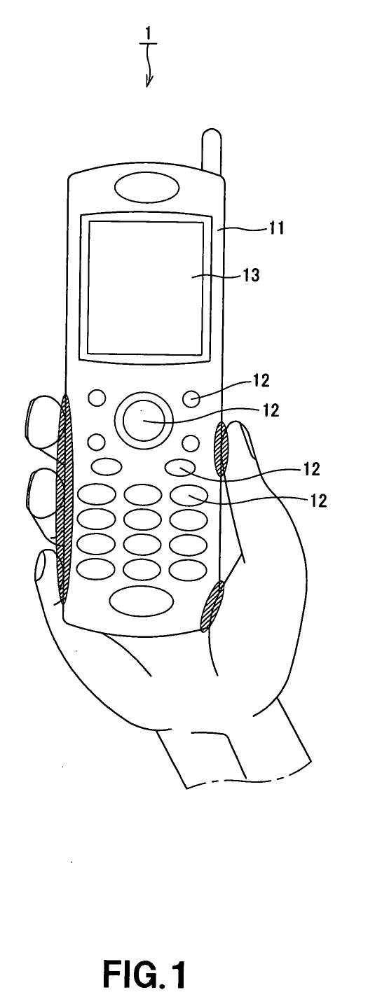

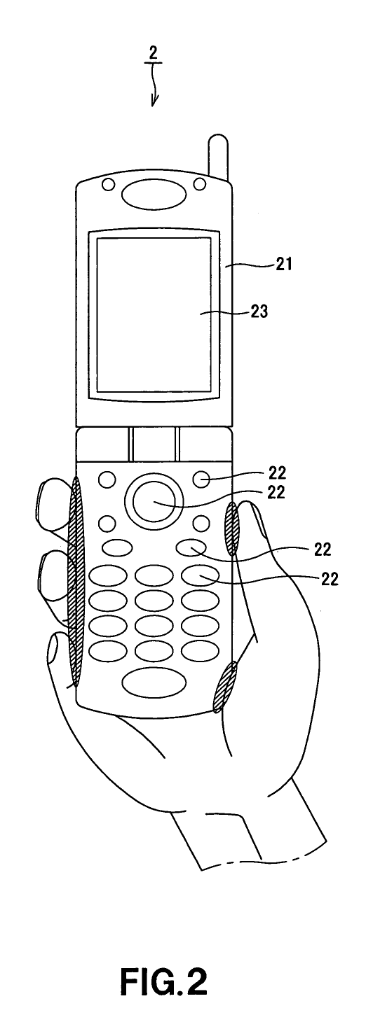

FIGS. 1 and 2 show, in a model form, the state where user grasps the mobile telephone by finger. The regions indicated by slanting lines in the figures are portion with which finger or palm of user come into contact by ordinary use. FIG. 1 shows the example of non-foldable type mobile telephone 1, and FIG. 2 shows the example of foldable type mobile telephone 2. At front face portions 11, 21 of the outer surfaces of casings constituting the telephone bodies, there are provided operation input buttons 12, 22 and display screens 13, 23 for executing functions of these mobile telephones 1, 2.

Galvanic Skin Reflex or Galvanic Skin Response (GSR) is bioindex used also in the so-called polygraph, and utilizes that electric resistance of skin changes by sweating. In order to measure GSR, it is necessary to detect Galvanic Skin Reflex (Response) between at least two points on the skin. In general, there is employed a technique in which electrode is caused to be in contact with the portion between two points of finger or palm to allow very weak current to flow to detect change quantity of resistance, etc. In view of the above, in the case of the mobile telephone, when the plan surface where display screen which displays guide display and information for operation is provided is caused to be front face portion of the casing as a position where measurement can be successively made even if explicit acquiring operation is not performed when user uses the mobile telephone by ordinary way of use at the time of operation such as calling (talking), and/or mail input, etc., GSR sensor is provided at the side surface portion of the outer peripheral side, the corner portion that the front face portion and the side face portion form, or the surface of the operation input button of the mobile telephone.

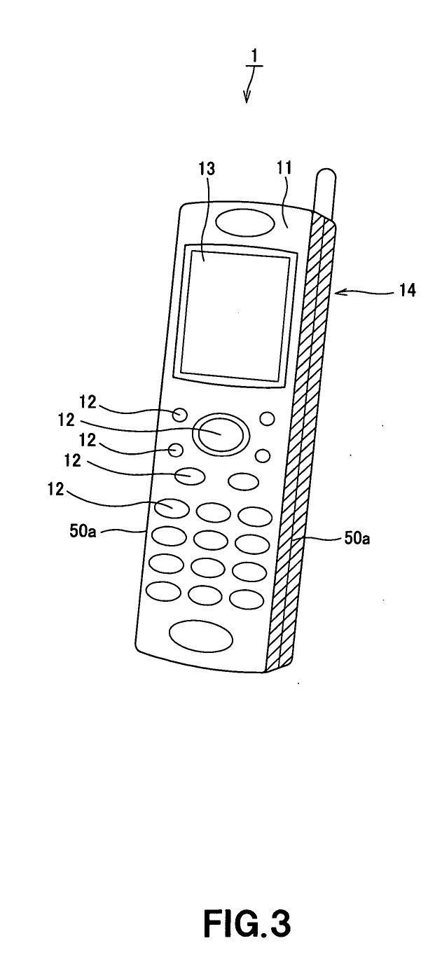

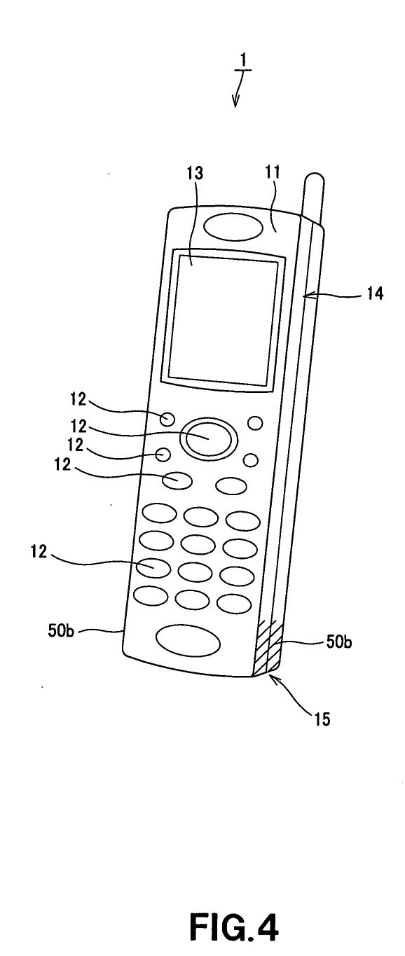

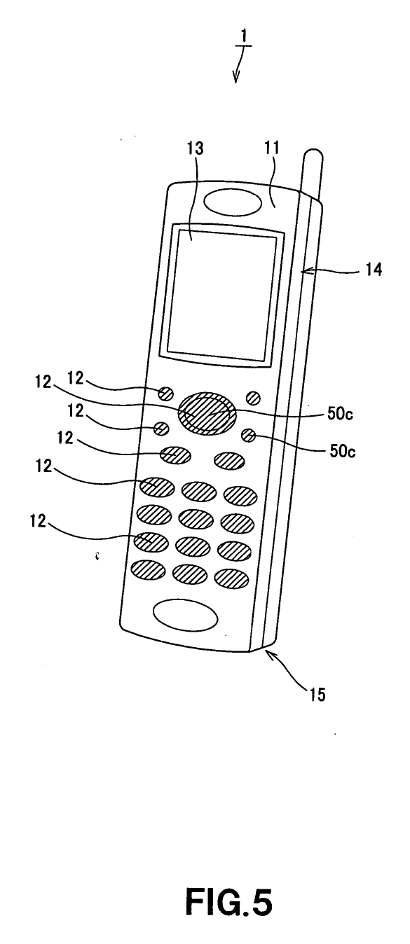

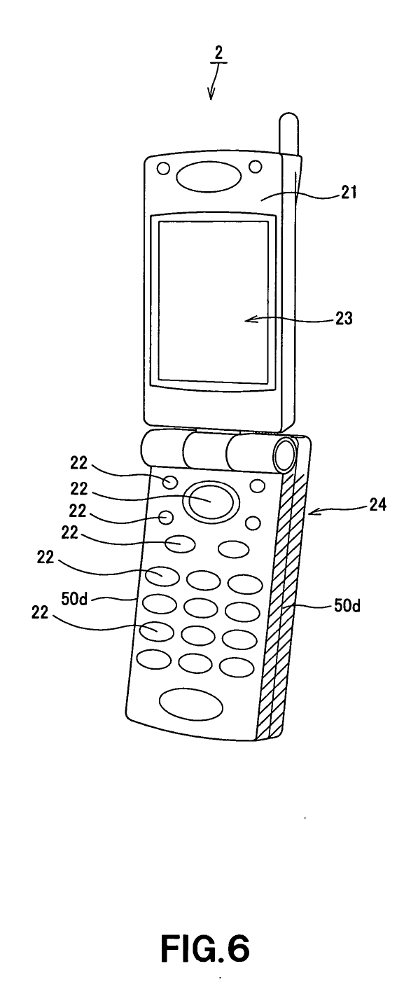

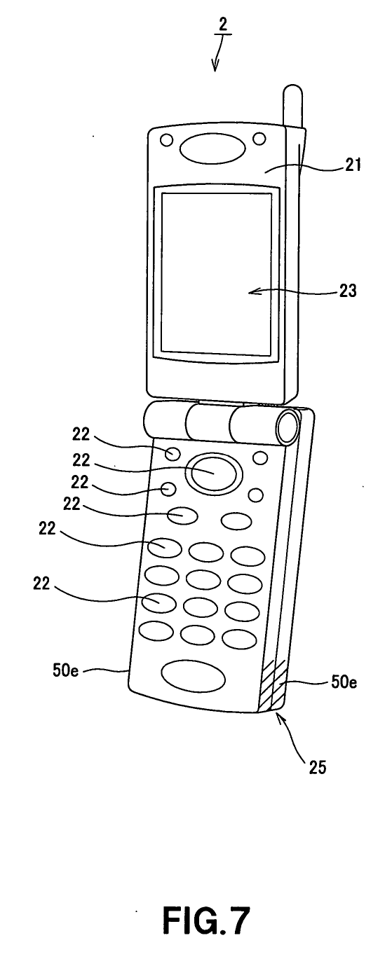

The example where GSR sensors 50a are attached to side surface sides 14 of the outer peripheral side of the non-foldable mobile telephone 1 is shown in FIG. 3, the example where GSR sensors 50b are attached to corner portions 15 of the outer peripheral side of the non-foldable type mobile telephone 1 is shown in FIG. 4, and the example where GSR sensors 50c are attached on the operation surface with which finger comes into contact of the operation input buttons 12 of the non-foldable type mobile telephone 1 is shown in FIG. 5. Moreover, the example where GSR sensors 50d are attached to side surface portions 24 of the outer peripheral sides of the foldable type mobile telephone 2 is shown in FIG. 6, the example where GSR sensors 50e are attached to corner portions 25 of the outer peripheral sides of the foldable type mobile telephone 2 is shown in FIG. 7, and the example where GSR sensors 50f are attached on the operation surfaces where finger comes into contact with operation input buttons 22 of the foldable type mobile telephone 2.

The components indicated by the same reference numerals of the GSR sensors 50 shown in FIGS. 3 to 6 represent a pair of sensors, wherein one sensor is cathode and the other sensor is anode. In the case where the GSR sensor 50 is provided on the operation input button, conductive material is used on the operation input button surface. Moreover, two electrodes spaced by a predetermined distance may be provided on single key so that a pair of sensors are provided.

Moreover, GSR sensors 50 may be provided at both the side surface portions 14 shown in FIG. 3 and the corner portions 15 of the outer peripheral sides of the casing shown in FIG. 4. Further, the GSR sensors 50 may be provided on the operation surfaces of the operation input buttons 12. In this case, e.g., the GSR sensor 50a provided at the side surface portion 14 may be anode, and the GSR sensor 50b provided at the corner portion 15 may be cathode. There may be employed a configuration in which the relationship between the sensor and electrode is opposite to the above. Moreover, GSR sensors 50a, 50b provided at the outer peripheral surface side of the casing are caused to be electrode of one polarity and GSR sensors 50c provided on the operation input buttons disposed on the casing are caused to be electrode of the other polarity, thereby making it possible to detect GSR at the portion between finger by which the operation button is pushed down and palm in contact with the GSR sensor of the outer edge when user pushes down the operation button. It is to be noted that whether electrode provided at an arbitrary portion is anode or cathode can be selected in accordance with optimum measurement distance of GSR.

By providing GSR sensors as described above, when user uses the mobile telephone, it is possible to acquire GSR serving as bioindex by itself by action to grasp such mobile telephone by finger. Moreover, even in the case where user grasps the mobile telephone by any one of right hand and left hand, GSR can be detected.

It is said that the body temperature, particularly the body temperature of the peripheral portion such as finger tip depends upon stress or comfortable/uncomfortable state. For example, when the human being is placed in uncomfortable state generally by stress, circulation of blood of vascular tract of the peripheral portion becomes bad so that temperature (body temperature) is partially lowered. In view of the above, temperature sensors for measuring temperature difference are disposed on the mobile telephone surface to measure temperature difference between the finger tip portion and palm, thereby making it possible to recognize comfortable/uncomfortable state or stress state, etc. of the human being. Similarly to the GSR sensor, temperature sensor may be disposed at the position which has been explained with reference to FIGS. 3 to 6. In the case where the temperature sensors are provided at the non-foldable type mobile telephone 1, these temperature sensors are provided on the side surface portions 14 of the casing, thus to detect finger tip temperature by one sensor, and to detect palm temperature by the other telephone. In addition, one sensor may be provided on the side surface portion 14 of the casing, and the other sensor may be provided at the corner portion of the outer peripheral side of the casing.

In the case where the temperature sensors are provided on the operation surfaces with which finger comes into contact of the operation input buttons, temperature sensor is provided also on the outer peripheral surface of the casing. In this case, finger tip temperature is detected by the sensor provided on the operation input button, and palm temperature is detected by the sensor provided at the casing side. It should be noted that in the case where the temperature sensor is disposed on the outer peripheral surface of the casing, heat insulating processing and/or circuit arrangement within the mobile telephone are changed for the purpose of preventing influence of heat produced from the mobile telephone body.

Subsequently, explanation will be given in connection with the case where mobile telephone is used to detect pulse wave of user as bioindex. It can be considered that pulse wave attempted to detect in this example is equivalent to heartbeat although phase somewhat shifts. In strained or excited state, pulse wave (speed) becomes fast. In stable state, pulse wave (speed) becomes slow. Ordinarily, pulse wave can be detected by optical pulse wave sensor. The optical pulse wave sensor employs a technique of irradiating rays of test light having a specific wavelength from the finger tip nail side to detect rays of transmitted light at the finger tip midsection side. When such a technique is employed, blood oxygen saturation level (SPO2) can be also acquired at the same time. However, in the optical pulse wave sensor, in order to continuously measure stable pulse waves, it is necessary to grasp the mobile telephone in the state where the positional relationship of the finger tip (nail side), the light emitting portion, the finger tip (midsection side) and the light receiving portion is stable to some degree. In view of the above, in mobile telephone 7 which will be explained in this practical example, pulse wave sensor portion comprising pulse wave sensor is provided at a portion of the casing constituting the telephone body.

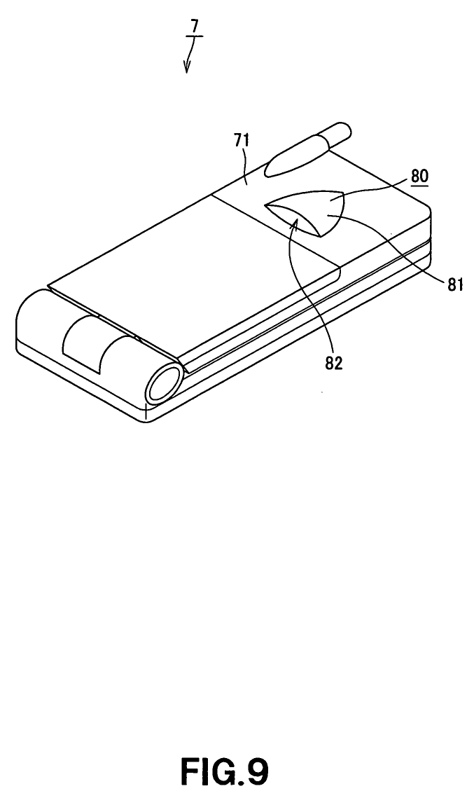

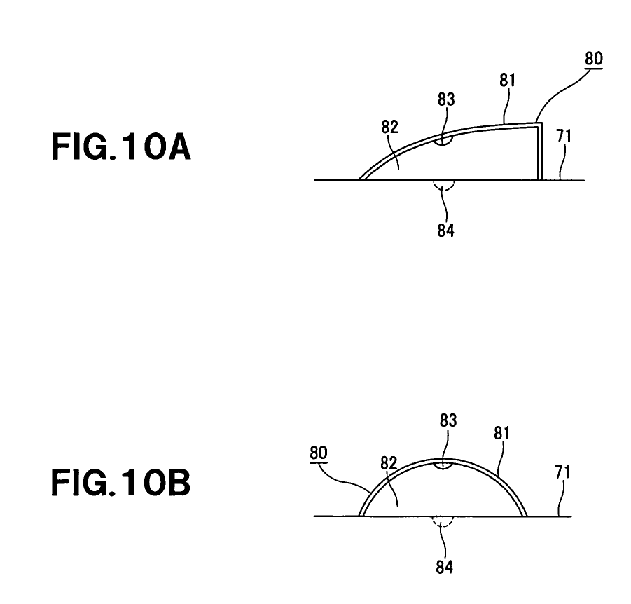

The mobile telephone 7 comprising pulse wave sensor portion 80 is shown in FIG. 9. Moreover, the cross section in which the pulse wave sensor portion 80 is cut in length side direction of the mobile telephone 7 is shown in FIG. 10A, and the cross section in which the pulse wave sensor portion 80 is cut in short side direction of the mobile telephone 7 is shown in FIG. 10B. At the mobile telephone 7, pulse wave sensor portion 80 is provided at a rear face portion 71 of the casing in which display screen serving to display guide display and information for operation is provided on the surface thereof. As shown in FIGS. 10A, 10B, the rear face portion 71 of the casing comprises a finger holding cover 81 having internal shape curved so as to take substantially the same shape as the finger tip shape of user, and a finger tip insertion portion 82 formed between the finger holding cover 81 and the rear face portion 71 to constitute the pulse wave sensor portion 80. The inside surface of the finger holding cover 81 comprises a light emitting portion 83 of the optical pulse wave sensor, wherein a light receiving portion 84 as pulse wave detecting means is provided at the position opposite to the light emitting portion 83 of the rear face portion 71. The light emitting portion 83 is disposed at a position where rays of test light having a specific wavelength can be irradiated substantially onto the finger tip nail upper portion when user inserts finger tip. Rays of test light which have been emitted at the light emitting portion 83 are detected by the light receiving portion 84 disposed at the middle section side of finger when user inserts finger tip as rays of light which have been transmitted through the finger inside.

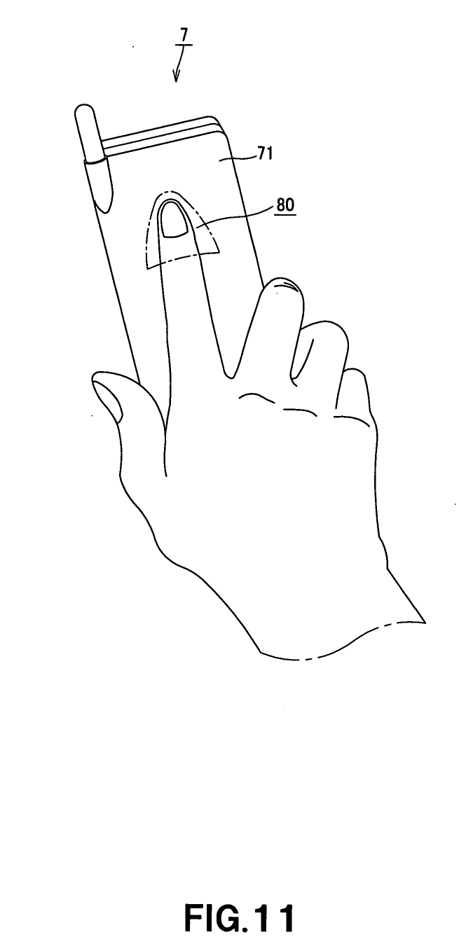

As shown in FIG. 11, if user grasps mobile telephone including pulse wave sensor 80 of such a structure by most popular method, there results the state where when user grasps the mobile telephone 7, the forefinger is inserted into the finger chip insertion portion 82 of the pulse wave sensor portion 80 without disagreement of feeling. Accordingly, for a time period during which user is using the mobile telephone 7, it is possible to perform stable measurement of pulse wave.

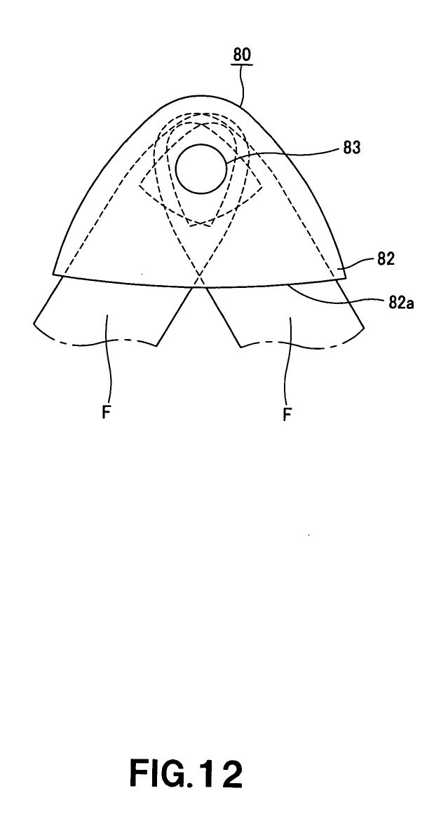

There is individual difference when user uses the mobile telephone depending upon the state where he grasps it by left or right hand. Even in the case of the same person, it is general that way of grasp by finger varies in dependency with calling or mail input. For this reason, as shown in FIG. 12, a finger insertion opening portion 82a of the pulse wave sensor portion 80 is widened in a sector form to devise opening shape of the finger tip insertion portion 82 such that the light emitting portion 83 is positioned substantially at the center of the finger nail even if finger F is inserted at an any angle. Thus, it is possible to comply with, e.g., unevenness of finger tip insertion angle due to difference of hand which grasps the mobile telephone 7. Moreover, the finger holding cover 81 forms curved surface and takes a shape such that the diameter is gradually reduced according as distance from the finger insertion opening portion toward long side direction is increased. For this reason, the degree of reduction of diameter is lowered to thereby broaden the space of the finger tip insertion portion 82. Thus, it is possible to absorb individual difference of size of finger.

In regard to the point where the pulse wave sensor portion 80 is provided so that the rear face portion 71 of the finger holding cover 81 is swelled out in a manner projected therefrom, the portion in which, e.g., the light receiving portion 83 is disposed is alloed to be recessed portion caused to be in conformity with finger shape to thereby suppress projection height from the rear face portion of the finger holding cover 81 to also have ability to realize thin structure.

It is to be noted that the mobile telephone 7 in which pulse wave sensor portion 80 is provided may be non-foldable mobile telephone, or may be foldable mobile telephone.

### (2) Second Embodiment

When biosensor for acquiring bioindex of user is attached to an input equipment that user operates in the state where he directly grasps it such as mobile telephone, remote controller, controller of television game machine, and/or mouse used as input means of computer, etc., measurement of bioindex which is tacit and is non-invasive/assaultive to user can be made. Particularly, since heartbeat, pulse wave, SPO2 (blood oxygen saturation level), skin temperature, Galvanic Skin Reflex and/or Galvanic Skin Response are bioindex which can be detected through the skin surface, even if operation for detection is not performed explicitly and temporarily, user grasps it by finger in use so that such bioindex can be acquired. If sensor position is devised, measurement can be made if ordinary use is performed without necessity of changing manner of holding or grasping the device. As bioindex which can be detected through skin surface, there are mentioned sweating, MV (Micro Vibration) and/or myoelectric potential, etc.

In the second embodiment, there are proposed a device and a method in which plural sensors are provided at an equipment utilizing bioindex of user to thereby have ability to detect bioindex data stably and with high accuracy. Initially, whether any data is used among bioindex data which have been acquired by the plural sensors is determined.

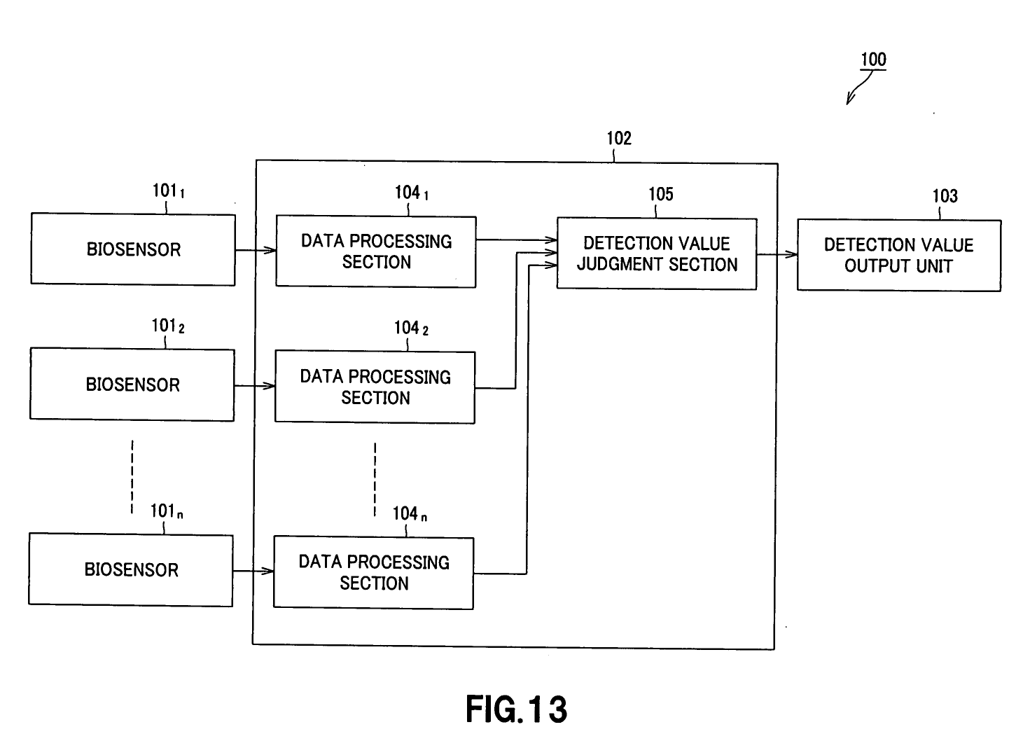

The fundamental configuration of a bioindex detecting apparatus of this embodiment will be explained by using FIG. 13. There is employed an approach to select stable value and value having high reliability among output values of biosensors disposed at plural portions, or to perform integrating processing of these values, thus to realize reliable data detection. Electronic equipment to be attached and attachment position thereof will be described later.

As shown in FIG. 13, the bioindex detecting apparatus 100 comprises biosensors 101₁, 101₂, . . . , 101ₙ, a detection value selector unit 102 for selecting any one of detection values which have been detected by these biosensors to output the selected detection value, and a detection value output unit 103 for outputting a detection value which has been selected by the detection value selector unit 102. Further, the detection value selector unit 102 comprises data processing sections 104₁, 104₂, . . . , 104ₙ for processing detection values which have been detected by the respective biosensors, and a detection value judgment section 105 for selecting an optimum value from detection values which have been processed by the data processing sections, wherein respective sections are caused to undergo supervisory control by control section (not shown). Controls of the respective sections may be performed by the control section of electronic equipment in which the bioindex detecting apparatus 100 is provided.

From the detection value output unit 103, detected bioindex is sent to the electronic equipment in which the bioindex detecting apparatus 100 is provided. The outputted bioindex is used at respective electronic equipments. There may be employed use purpose such that, e.g., if the electronic equipment is mobile telephone, detected bioindex of user is transmitted to the opposite side of communication, and if the electronic equipment is remote controller of the air conditioner, temperature setting is made in accordance with detected bioindex of user, or the like.

The biosensor 101 is a sensor for detecting bioindex which can be detected by contact of the skin surface, and a sensor for heartbeat, pulse wave, SPO2 (blood oxygen saturation level), skin temperature, Galvanic Skin Reflex or Galvanic Skin Response may be applied for this purpose. Plural sensors selected therefrom are provided on an electronic equipment surface such that bioindex can be acquired even if user does not change manner of holding again when user grasps the electronic equipment by finger in ordinary use, or finger comes into contact therewith. Moreover, while the biosensors 101 are respectively the same sensor in the example shown in FIG. 13, there may be employed different kinds of biosensors for detecting the same bioindex by different techniques, or different bioindices may be detected by different kinds of biosensors.

In this example, the data processing sections 104 in the detection value selector unit 102 calculate SN (signal-to-noise) ratios of respective output values of bioindex which have been detected by the biosensors. The SN ratios which have been calculated with respect to output values from the respective sensors are sent to the detection value judgment section 105. The data processing sections 104 may be processing sections for calculating detection levels of detected output values or auto-correlation functions of detected output values also in addition to the SN ratio. In the case where respective plural biosensors 101 are the same biosensor for detecting the same bioindex, the detection value judgment section 105 may select, as output value, value which has been detected substantially as the same value by plural sensors, or may calculate average value of values detected by respective biosensors to select the average value thus calculated as an output value. In addition, in the case where detection value is caused to undergo digital processing, error rates may be compared to select detection value having small error.

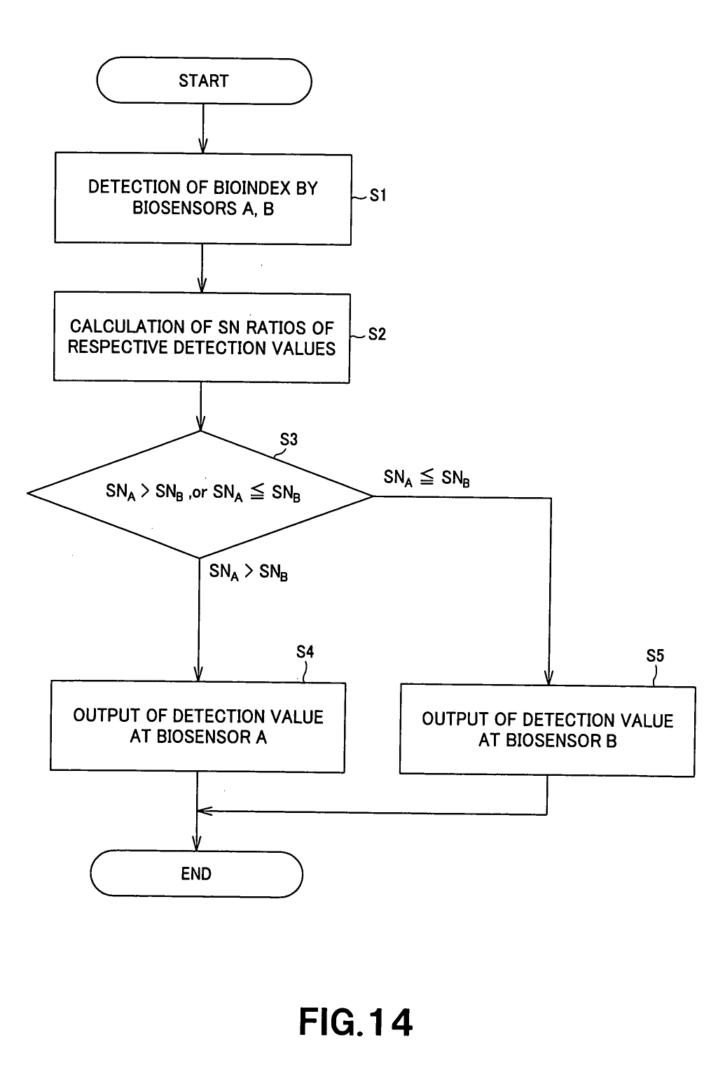

Subsequently, the processing in which the bioindex detecting apparatus judges reliability of detection values at respective biosensors to select an optimum value is shown in FIG. 14. FIG. 14 shows the processing for selecting detection values which have been detected by two biosensors of the biosensor A and the biosensor B. The biosensor A and the biosensor B are the same biosensor for detecting the same bioindex.

As step S1, the biosensor A and the biosensor B detect bioindex values. The detection values of the bioindex are caused to undergo data processing at step S2 so that SN ratios are determined. Then, at step S3, SN ratios are compared with each other. Here, whether or not any one of SN ratio of detection value at the biosensor A (SNA) and SN ratio of detection value at the biosensor B (SNB) is high is discriminated. Namely, if SNA>SNB, detection value at the biosensor A is selected. At step S4, value which has been detected at the biosensor A is sent to the detection value output section. On the other hand, if SNA≦SNB, detection value at the biosensor B is selected. At step S5, value which has been detected at the biosensor B is sent to the detection value output unit.

At the step S4 of FIG. 14, data of the biosensor A having SN higher than that of the biosensor B is outputted. Since data having high SN ratio is selected at all times in accordance with this processing, biosensors to be selected change without interruption in a manner of, e.g., A→B→A→A→B with time change. It is considered that the processing shown in FIG. 14 is effective particularly in the case where one detection value is obtained by one sensor, e.g., in the case where body temperature (finger tip temperature and palm temperature) is detected, etc.

Moreover, as another example, in the case where threshold value is provided at detection value so that the biosensors A and B both have not a predetermined SN or more, output is not provided. In this case, if that threshold value is caused to be SNSH, SNA>SNB≧SNSH or SNB≧SNA≧SNSH is judged at the step S3.

Furthermore, in the case where threshold value is provided in difference between detection values of both biosensors so that unevenness between detection values of the both biosensors is large, output may not be provided. In this case, at the step S3, whether |SNA−SNB|≧SNSH and SNA>SNB, or |SNA−SNB|≧SNSH and SNB≧SNA is judged.

As practical example of the embodiment, the state where mobile telephone is used to detect bioindex of user who utilizes the mobile telephone will be described below. The bioindex detected here is bioindex which can be relatively easily acquired from user who uses mobile telephone, and there are mentioned, e.g., Galvanic Skin Reflex, Galvanic Skin Response, pulse wave and/or body temperature (skin temperature) of user.

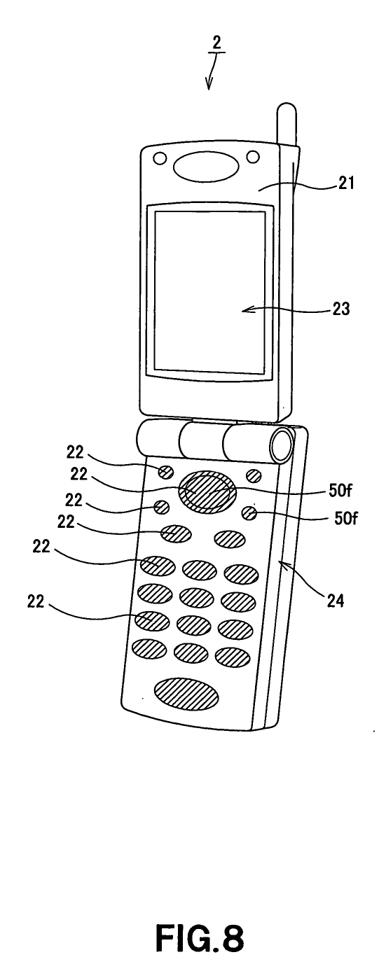

It is to be noted that since practical example of attachment with respect to the mobile telephone of GSR sensor 50 is similar to that explained by using FIGS. 3 to 8 in the first embodiment, the FIGS. 3 to 8 are referred to thereby omit further detailed explanation.

Initially, for the purpose of determining GSR, it is necessary to detect Galvanic Skin Reflect (Response) between at least two points on the skin. While there is generally employed a technique in which electrode is caused to be in contact with the portion between two points of finger or palm to allow very weak current to flow to detect change quantity of resistance, etc., plural pairs of sensors each comprised of a pair of electrodes are provided in this example to select output from an optimum sensor pair.

Respective GSR sensors 50a, 50b, 50c, 50d, 50e, 50f provided on the outer peripheral surface of the side surface portion, etc. of the casing constituting the telephone body, and or the operation surface of the operation input button thereof are divided into plural regions to constitute respective one sensors with respective region pairs being as one set. In the examples shown in FIGS. 3 to 8, respective sets of sensors are constituted by pairs of GSR sensors 50a, 50b, 50c, 50d, 50e, 50f indicated by the same reference numerals.

Here, one of each paired GSR sensors 50a, 50b, 50c, 50d, 50e, 50f constitutes cathode, and the other GSR sensor constitutes anode.

In this example, sensor provided on the operation surface with which finger comes into contact of the operation input button is constituted by depositing conductive material on the surface of the operation input button. In addition, two electrodes may be provided in the state spaced by a predetermined distance on one operation input button to thereby a pair of GSR sensors.

By providing the GSR sensors 50a, 50b, 50c, 50d, 50e, 50f in a manner as shown in the previously mentioned FIGS. 3 to 8, when user uses the mobile telephone, GSR serving as bioindex can be acquired by itself by the action to grasp the mobile telephone 1 or 2 as previously described. Moreover, by providing the GSR sensors 50a, 50b, 50c, 50d, 50e, 50f in a manner as shown in FIGS. 3 to 8, even in the case where user grasps the mobile telephone 1 or 2 by any one of right and left hands, it is possible to perform detection of GSR.

Similarly to the GSR sensors, temperature sensors may be disposed at the positions which have been explained with reference to FIGS. 3 to 6. In the case where the temperature sensors are provided at the non-foldable type mobile telephone 1, there is employed, similarly to the GSR sensor, an approach in which plural temperature sensors are provided at the side surface portions 14 of the casing to detect temperature of finger tip by one group of sensors, and to detect temperature of palm by the other group of sensors. Moreover, there may be also employed a configuration in which one group of sensors are provided at the side surface portions 14 of the casing, and the other group of sensors are provided at the corner portions of the outer peripheral side of the casing.

In the case where temperature sensors are provided on the operation input button, one of the pair of sensors is provided at the operation input button side, and the other sensor is provided at the outer peripheral surface side of the casing to constitute a set of temperature sensors. In this case, finger tip temperature is detected by the sensor provided on the operation surface of the operation input button, and palm temperature is detected by the sensor provided at the casing side. It is to be noted that in the case where the temperature sensor is disposed on the surface of the casing, heat insulating processing and/or circuit arrangement within the mobile telephone are changed for the purpose of preventing influence of heat produced from the mobile telephone body.

Also in the case where the mobile telephone is used to detect pulse wave of user as bioindex, pulse wave sensor similar to the first embodiment is used.

While pulse wave sensors may be provided at plural portions in this practical example, there may be also employed a configuration, as has been explained with reference to FIGS. 9 to 12, in which single pulse wave sensor is provided, and GSR sensor or temperature sensor may be further provided.

As explained also in the above-described first embodiment, plural biosensors may be the same kind of sensors, or may be different kind of biosensors also in this embodiment.

As described above, biosensors are provided at plural portions of the surface of the electronic equipment such as mobile telephone, etc. to acquire bioindex values from the plural portions to thereby eliminate unevenness of detection values taking place by difference of manner of holding or grasping of the electronic equipment or difference of the state of skin surface, etc. thus to have ability to improve reliability of detection values. Moreover, there are some mobile telephones capable of performing calling operation (talking) as telephone and further having mail transmit/receive function. In such a case, it is also possible to comply with difference of manner holding or grasping in use as in the case of the time of calling and the time of preparing mail. Further, in future, it is predicted that there will appear electronic equipments utilizing biometrics in the field except for authentication. It cannot be said that the bioindices are all directed to bioindices in which input operation (authentication operation) is explicitly and temporarily performed as in the case of fingerprint authentication, for example. In accordance with this embodiment, it is possible to acquire bioindex more simply and stably. Thus, it is possible to improve reliability of bioindex values acquired at the biometrics application equipments.

It is to be noted that while the invention has been described in accordance with certain preferred embodiments thereof illustrated in the accompanying drawings and described in the above description in detail, it should be understood by those ordinarily skilled in the art that the invention is not limited to the embodiments, but various modifications, alternative constructions or equivalents can be implemented without departing from the scope and spirit of the present invention as set forth and defined by the appended claims.

### INDUSTRIAL APPLICABILITY

While the present invention has been explained by taking an example of mobile telephone as portable or mobile electronic equipment, the present invention can be applied, without being limited to mobile telephone, also to any input devices comprising input unit such as operation button, etc. which are used in the state grasped by finger and serving to perform input of operation instructions and/or various information. For example, in recent years, digital televisions of the interactive (bidirectional) communication system, etc. have appeared. Bioindex detecting means may be disposed, in the above-described manner, at remote controller of multi-functional television and/or audio-visual equipment, and/or controller of television game machine, etc. Moreover, bioindex detecting means is provided within a region including position with which finger comes into contact of the surface of body to be operated that user grasps by finger in use such as control (steering) lever of ship or airplane, etc. and/or handle of automotive vehicle, etc. to thereby have ability to successively acquire bioindices through skin surface from contact when user grasps the body to be operated for the purpose of performing operation without allowing user to explicitly execute acquiring operation of bioindex. In addition, if various bioindices can be acquired, there can be created new entertainment use purpose and/or new technical use purpose in which biometrics technology of new bioindies is applied. For example, the present invention can be applied to information communication equipments in which detected bioindex of user is transmitted to the opposite side of communication, or information output corresponding to biostate calculated from bioindex of user is provided, and/or life equipments such that temperature setting is made in accordance with detected bioindex of user, tec.

1. An input device comprising:
bioindex detecting means provided within a region including a holding position of the surface of a body to be operated, that user holds in use, and for detecting, for a time period during which user grasps the body to be operated, bioindex of the user through skin of the user; and
bioindex analyzing means for analyzing bioindex which has been detected by the bioindex detecting means.

2. The input device according to claim 1,
wherein the bioindex is at least one of sweating, heartbeat, pulse wave, Galvanic Skin Reflex, Galvanic Skin Response, MV (Micro Vibration), myoelectric potential and SPO2 (blood oxygen saturation level), and combination of these bioindices.

3. The input device according to claim 1,
wherein the bioindex detecting means is detecting means for detecting Galvanic Skin Reflex or Galvanic Skin Response between predetermined two points of palm of one hand of user.

4. The input device according to claim 1,
wherein the bioindex detecting means is pulse wave detecting means for detecting pulse wave of user.

5. The input device according to claim 1,
wherein the bioindex detecting means is temperature detecting means for detecting body temperature of user.

6. The input device according to claim 5,
wherein the temperature detecting means is composed of finger tip temperature detecting means for detecting finger tip temperature provided at a position with which finger tip comes into contact when the finger tip temperature detecting means is grasped by finger of the user, and palm temperature detecting means provided at a position with which palm of the user comes into contact and for detecting palm temperature.

7. The input device according to claim 1, including:
the plural bioindex detecting means,
the input device further comprising:
selector means for selecting at least one bioindex information from bioindex information which have been detected by the plural bioindex detecting means,
wherein the bioindex analyzing means serves to analyze bioindex information which has been selected by the selector means.

8. The input device according to claim 7,
wherein the selector means serves to compare signal-to-noise ratios of output values which have been detected by the plural bioindex detecting means to select an output value having value of higher signal-to-noise ratio.

9. The input device according to claim 7,
wherein the selector means serves to compare detection levels of output values which have been detected by the plural bioindex detecting means to select an output value having higher detection level.

10. The input device according to claim 7,
wherein the selector means serves to compare auto-correlation functions of output values which have been detected by the plural bioindex detecting means to select an output value in which correlation has been taken to more degree.

11. The input device according to claim 7,
wherein the selector means serves to select one output from outputs from the plural bioindex detecting means.

12. The input device according to claim 7,
wherein the selector means serves to select, as an output value, a value which has been detected substantially as the same value at the plural bioindex detecting means.

13. The input device according to claim 7,
wherein the selector means serves to select, as an output value, an average value obtained by averaging values detected at the respective bioindex detecting means.

14. The input device according to claim 7,
wherein the respective plural bioindex detecting means are similar bioindex detecting means for detecting the same bioindex.

15. The input device according to claim 7,
wherein the respective plural bioindex detecting means are different kinds of bioindex detecting means for detecting the same bioindex by different techniques.

16. The input device according to claim 7,
wherein the respective plural bioindex detecting means are different kinds of bioindex detecting means for detecting different bioindices.

17. The input device according to claim 7,
wherein the input device is provided at an operation input unit of any one of electronic equipments including personal computer, television image receiver, video and/or audio signal recording and/or reproducing device and air conditioner.

18. The input device according to claim 7,
wherein the input device is provided at controller for television game machine.

19. The input device according to claim 7,
wherein each of the plural bioindex detecting means is provided at a control or steering unit that user holds in control or steering at any one of machines to be controlled including automotive vehicle, train, airplane, ship and industrial machinery.

20. An input method including:
a bioindex detection step of detecting, by detecting means provided within a region including a holding position of the surface of a body to be operated, that user holds in use, bioindex of the user through skin of the user for a time period during which the user holds the body to be operated; and
a bioindex analysis step of analyzing bioindex which has been detected at the bioindex detection step.

21. The input method according to claim 20,
wherein the bioindex is at least one of sweating, heartbeat, pulse wave, skin temperature, Galvanic Skin Reflex, Galvanic Skin Response, MV (Micro Vibration), myoelectric potential and SPO2 (blood oxygen saturation level), or combination of these bioindices.

22. The input method according to claim 20,
wherein the bioindex detection step consists of plural bioindex detection steps,
the input method including:
a selection step of selecting at least one bioindex information from bioindex information which have been detected at the plural bioindex detection steps; and
a bioindex analysis step of analyzing bioindex information which has been selected at the selection step.

23. The input method according to claim 22,
wherein the respective plural bioindex detection steps detect the same bioindex.

24. The input method according to claim 22,
wherein the respective plural bioindex detection steps detect the same bioindex by different techniques.

25. The input method according to claim 22,
wherein the respective plural bioindex detection steps detect different bioindices.

26. An electronic equipment including an input unit comprising:
bioindex detecting means provided within a region including a holding position of the surface of a body to be operated, with which finger comes into contact when user grasps the body to be operated through the finger in use, and for detecting bioindex of the user through skin of the user for a time period during which the user grasps the body to be operated by finger; and
bioindex analyzing means for analyzing bioindex which has been detected by the bioindex detecting means.

27. The electronic equipment according to claim 26,
wherein the bioindex is at least one of sweating, heartbeat, pulse wave, skin temperature, Galvanic Skin Reflex, Galvanic Skin Response, MV (Micro Vibration), myoelectric potential and SPO2 (blood oxygen saturation level), and combination of these bioindices.

28. The electronic equipment according to claim 26,
wherein the bioindex detecting means is detecting means for detecting Galvanic Skin Reflex or Galvanic Skin Response between predetermined two points of palm of one hand of user.

29. The electronic equipment according to claim 28,
wherein display means for displaying guide display for operation and information is provided at the front face portion of a casing,
the detecting means being provided at the side surface portion of the casing.

30. The electronic equipment according to claim 28, comprising:
operation means for an operation input,
wherein the detecting means is provided at a position with which finger of user comes into contact of the surface of the operation means.

31. The electronic equipment according to claim 28,
wherein the detecting means is provided at the corner portion of the casing.

32. The electronic equipment according to claim 28,
wherein the bioindex detecting means is pulse wave detecting means for detecting pulse wave of user.

33. The electronic equipment according to claim 32,
wherein display means for displaying guide display for operation and information is provided at the front face portion of the casing, and
the pulse wave detecting means is provided at the rear face portion opposite to the front face portion of the casing.

34. The electronic equipment according to claim 33,
wherein a detection portion comprising a finger holding cover having internal surface shape curved so as to take substantially the same shape as finger tip shape of the user, and a finger chip insertion portion formed between the finger holding cover and the rear face of the casing is provided at the rear face portion side of the casing,
light emitting means being provided at the inner surface of the finger holding cover, light receiving means as the pulse wave detecting means being provided at the rear face of the casing opposite to the light emitting means.

35. The electronic equipment according to claim 26,
wherein the bioindex detecting means is temperature detecting means for detecting body temperature of user.

36. The electronic equipment according to claim 35,
wherein the temperature detecting means is composed of finger tip temperature detecting means provided at a position with which finger comes into contact when the temperature detecting means is grasped by the finger of the user and for detecting finger chip temperature, and palm temperature detecting means provided at a position with which palm of the user comes into contact and for detecting palm temperature.

37. The electronic equipment according to claim 36, comprising:
display means serving to display guide display for operation and information at an outer casing front face portion,
wherein one of the temperature detecting means is provided at the side surface portion with respect to the outer casing front face portion.

38. The electronic equipment according to claim 36, comprising:
operation means,
wherein the finger tip temperature detecting means is provided at a position with which finger of user comes into contact of the surface of the operation means.

39. The electronic equipment according to claim 36,
wherein the palm temperature detecting means is provided at the corner portion of the outer peripheral surface side of the casing.

40. The electronic equipment according to claim 36,
wherein a detecting portion comprising a finger holding cover having an internal surface shape curved so as to take substantially the same shape as finger tip shape of the user, and a finger tip insertion portion formed between the finger holding cover and the rear face of the casing is provided at the rear face portion side of the casing,
the finger tip temperature detecting means being provided at the rear face portion of the casing.

41. The electronic equipment according to claim 26, including:
the plural bioindex detecting means,
the electronic equipment further comprising selector means for selecting at least one bioindex information from bioindex information which have been detected by the plural bioindex detecting means,
wherein the bioindex analyzing means serves to analyze bioindex information which has been selected by the selector means.
